(19) United States
(12) Patent Application Publication
(10) Pub. No.: US 2025/0378931 A1
(43) Pub. Date: Dec. 11, 2025

(54) SYSTEMS AND METHODS FOR PREDICTING AN IMAGE ACQUISITION COMPLEXITY OF AN IMAGING EXAMINATION

(71) Applicant: KONINKLIJKE PHILIPS N.V., EINDHOVEN (NL)

(72) Inventors: SIVA CHAITANYA CHADUVULA, MALDEN, MA (US); OLGA STAROBINETS, NEWTON, MA (US); RANJITH NAVEEN TELLIS, TEWKSBURY, MA (US); EKIN KOKER, CAMBRIDGE, MA (US); SANDEEP MADHUKAR DALAL, WINCHESTER, MA (US); THOMAS ERIK AMTHOR, HAMBURG (DE); YUECHEN QIAN, LEXINGTON, MA (US)

(21) Appl. No.: 18/876,685

(22) PCT Filed: Jun. 15, 2023
(86) PCT No.: PCT/EP2023/066143

§ 371 Date: Dec. 19, 2024

Related U.S. Application Data
(60) Provisional application No. 63/356,515, filed on Jun. 29, 2022.

(51) Int. Cl. G16H 20/40; G16H 10/60; G16H 30/20; G16H 30/40; G16H 40/20; G16H 40/63; G16H 40/67; G16H 50/30

(52) U.S. Cl. CPC G16H 20/40; G16H 10/60; G16H 30/20; G16H 30/40; G16H 40/20; G16H 40/63; G16H 40/67; G16H 50/30

(57) ABSTRACT

An apparatus (1) for providing assistance during medical imaging examinations performed in imaging bays (3) using medical imaging devices (2) each having an imaging device controller (10) with a controller display (24′) includes a remote electronic processing device (12) operatively connected to receive data streams (17, 18, 34) from the imaging bay including a screen mirroring data stream (34) that carries content presented on the controller display and provide a natural language communication pathway (19) connecting the imaging bay and the remote electronic processing device. An electronic processor (14s) is programmed to perform a method (100) to assess complexity of an imaging examination identified by a scheduler (40) including acquiring data related to the upcoming medical imaging examination; determining a complexity of the upcoming medical imaging examination based on the acquired data; and outputting an alert (30) indicative of the determined complexity of the upcoming medical imaging examination.

The following relates generally to the imaging arts, remote imaging assistance arts, remote imaging examination monitoring arts, and related arts.

### BACKGROUND

Medical imaging, such as computed tomography (CT) imaging, magnetic resonance imaging (MRI), positron emission tomography (PET) imaging, fluoroscopy imaging, and so forth, is a critical component of providing medical care, and is used in a wide range of medical fields, such as cardiology, oncology, neurology, orthopedics, to name a few. The operator of the medical imaging device used to acquire the medical images is typically a trained technologists, while interpretation of the medical images is often handled by a medical specialist such as a radiologist. Interpretation of radiology reports or findings by the radiologist can be handled by the patient's general practitioner (GP) physician or a medical specialist such as a cardiologist, oncologist, orthopedic surgeon, or so forth.

Currently, diagnostic imaging is in high demand. As the world population ages, the demand for quick, safe, high quality imaging will only continue to grow, putting further pressure on imaging centers and their staff. Under such conditions, errors are unavoidable, but can be often costly. One approach for imaging centers to boost efficiency and grow operations at no extra labor costs is through a radiology operations command center (ROCC) system. Radiology operations command centers enable teams to work across the entire network of imaging sites, providing their expertise as needed and remotely assisting less experienced technologists in carrying out high quality scans. Remote technologists or experts can monitor the local operators of scanning procedures through cameras installed in the scanning areas (or from other sources, such as sensors (including radar sensors), console video feeds, microphones connected to Internet of Things (IoT) devices, and so forth. In addition, these sources can be supplemented by other data sources like Health-Level 7 (HL7), Digital Imaging and Communications in Medicine (DICOM), Electronic Health Record (EHR) databases, and so forth.

The remote technologist (i.e. “super-tech,” also referred to herein as an “expert tech” or a remote expert)) is expected to be concurrently assigned to assist a number of different imaging bays at different sites that may be spread out across different cities or different states. In practice, however, the super-tech can only be paying attention to a single imaging bay at any given time. The super-tech will typically be assisting local technologists who actively call for super-tech support. However, situations may arise in which the super-tech's assistance would be beneficial, but the local technologist is unaware of the need for super-tech assistance, or chooses not to call for such assistance.

Activities performed for image acquisition for MR exams can be broadly classified into preparation for scanning and actual scanning. Preparation for scanning involves several tasks including room cleaning (after the previous patient leaves), registering patient, setup of contrast, coil if needed etc. On the other hand, scanning is related to taking images of the patient by executing a set of sequences dictated by the clinical need of the patient. This activity involves tasks such as selection of technology (e.g., compressed sense), coil selection and their placement, add/repeat sequences based on several factors including patient motion and technologist expertise. The multitude of tasks within each activity and their dependencies directly affect the time taken to complete the exam, quality of the images obtained, safety of the patient. These factors in turn contribute to the complexity of image acquisition.

ROCC enables a remotely located expert technologist to provide real-time support to the technologist performing the two activities during the image acquisition. Prediction of this real-time support is crucial for planning and smooth running of MR operations. Complexity of the image acquisition is one of the important contributors for predicting the real-time support.

ROCC brings a virtual way of supporting local technologist in resolving their day-to-day issues in real-time. Currently, an expert technologist is dedicated to a set of MR rooms (e.g., three) within a facility. In this setting, a problem can arise if two or more local technologists require support at a given time, as this creates difficulties for not only allocating the expert technologists per MR room but also determining their operating hours/schedules. In some care facilities, operational practices such as few hours in the MR scanner schedule being reserved for complex image acquisition procedures every week are followed to balance the support across the day and available MR rooms. However, these solutions are prone to sub-optimal solutions as the complexity in the image acquisition not only depends on the expertise of local technologist but also patient and examination characteristics.

The following discloses certain improvements to overcome these problems and others.

### SUMMARY

In one aspect, an apparatus for providing assistance during medical imaging examinations performed in imaging bays using medical imaging device each having an imaging device controller with a controller display is provided. The system includes a remote electronic processing device operatively connected to receive one or more data streams from the imaging bay including at least a screen mirroring data stream that carries content presented on the controller display and to provide a natural language communication pathway connecting the imaging bay and the remote electronic processing device. An electronic processor is programmed to perform a complexity assessment method to assess complexity of an upcoming medical imaging examination identified by a scheduler. The method includes acquiring data related to the upcoming medical imaging examination; determining a complexity of the upcoming medical imaging examination based on the acquired data; and outputting an alert indicative of the determined complexity of the upcoming medical imaging examination.

In another aspect, a non-transitory computer readable medium stores instructions executable by at least one electronic processor to perform a method of providing assistance during a medical imaging examination performed using a medical imaging device is provided. The method includes acquiring data related to one or more imaging examinations; determining a complexity of the one or more imaging examinations; and outputting an alert indicative of the determined complexity of the one or more imaging examinations.

In another aspect, a method of providing assistance during a medical imaging examination performed using a medical imaging device includes acquiring data related to one or more imaging examinations; determining a complexity of the one or more imaging examinations; generating a prediction of one or more factors contributing to the determined complexity of the one or more imaging examinations; and outputting an alert indicative of the determined complexity of the one or more imaging examinations and of the predicted one or more factors.

One advantage resides in determining a potential complexity of an imaging examination.

Another advantage resides in estimating a likelihood of assistance for a technologist performing an imaging examination.

Another advantage resides in assisting a local technologist in determining whether to request assistance from a remote expert monitoring an imaging examination.

Another advantage resides in determining a potential complexity of an imaging examination based on an expertise of a technologist performing the examination and patient characteristics of a patient who is to undergo the imaging examination.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically shows an illustrative apparatus for providing remote assistance in accordance with the present disclosure.

FIG. 2 shows an example flow chart of operations suitably performed by the apparatus of FIG. 1.

### DETAILED DESCRIPTION

The following discloses an ROCC system, in which a prediction is made as to the imaging examination complexity. The prediction is made prior to commencement of the examination, and may be used for example to alert the remote expert that the upcoming examination has complexities that may require assistance, or to similarly alert the imaging technician the upcoming examination will present certain challenges. The current data-driven approach considers multiple factors including history of interactions between expert and local technologist and their associated context of the examination to estimate the complexity of the image acquisition involved. This way of estimation can help prioritize and focus day-to-day issues during the morning huddle which in turn can minimize operational disruptions.

Unlike traditional in-person support, ROCC platform promotes support remotely. It is possible that the local technologists face certain barriers in adopting to this way of working and may hesitate to call expert technologist. However, the prediction of complexity in image acquisition can help expert technologists to decide whether they need to passively monitor a particular examination or not. This allows expert technologists to intervene on a necessary basis. This is especially useful when expert technologist is taking control of multiple MR scanner rooms.

As disclosed herein, upcoming medical imaging examinations on the schedule are assessed as to complexity. The disclosed approach for examination complexity prediction relies on information gleaned from sources such as the HL7 messages, the examination order placed by the referring physician, the examination card (once generated by the radiology department), the patient electronic medical record, imaging technician education and experience records, and so forth. Relevant information for the complexity prediction may include, for example, imaging modality, imaged anatomy, the specific imaging sequences specified in the examination card, and so forth. Further information obtained from the ROCC and/or a Picture Archiving and Communication System (PACS) may include information on historical examinations such as total length, time for each imaging examination phase (e.g. patient preparation, patient loading, imaging data acquisition, contrast agent delivery (if appropriate), patient unloading, idle time between these various phases, and so forth.

To predict complexity, such information is collected for historical imaging examinations which can be partitioned into various examination types based on salient information such as examination type (suitably characterized by parameters such as modality, specific imaging sequences, et cetera), patient characteristics, the specific imaging technologist performing the examination or a technicians class segmented by experience (0-2 years, 2-5 years, . . . ), etc.

For each examination type, performance statistics for the historical imaging examinations falling in that class generated for various complexity metrics. The metrics may include, for example, average total examination time, average time for each phase, average idle time between successive phases, average number of rescans, average number of consultations with a remote expert, average obtained image quality, and so forth. The examination complexity of each upcoming medical imaging examination of that examination type is then determined based on its statistics.

Advantageously, this approach enables, for each examination type, both an overall examination complexity prediction and a prediction of which factors introduce the complexity. Hence, the imaging technician and/or remote expert can be informed as to both the overall complexity and the areas that may be particularly challenging.

If there is missing information that would ordinarily be used to predict complexity of a given examination, this can be handled in various ways, such as assigning an “average” value or the missing datum, or generating complexity prediction statistics for coarser examination types that omit that information. For example, a finer-grained examination type may be segmented by imaging technician experience, while a coarser-grained examination type may group all examinations together regardless of technician experience.

In some embodiments, the local technician and/or remote expert may provide feedback on the perceived complexity of an imaging examination at the end of the examination, and this information (e.g. the averaged perceived complexity) may constitute further statistical data for assigning a complexity prediction for future examinations of a particular examination type.

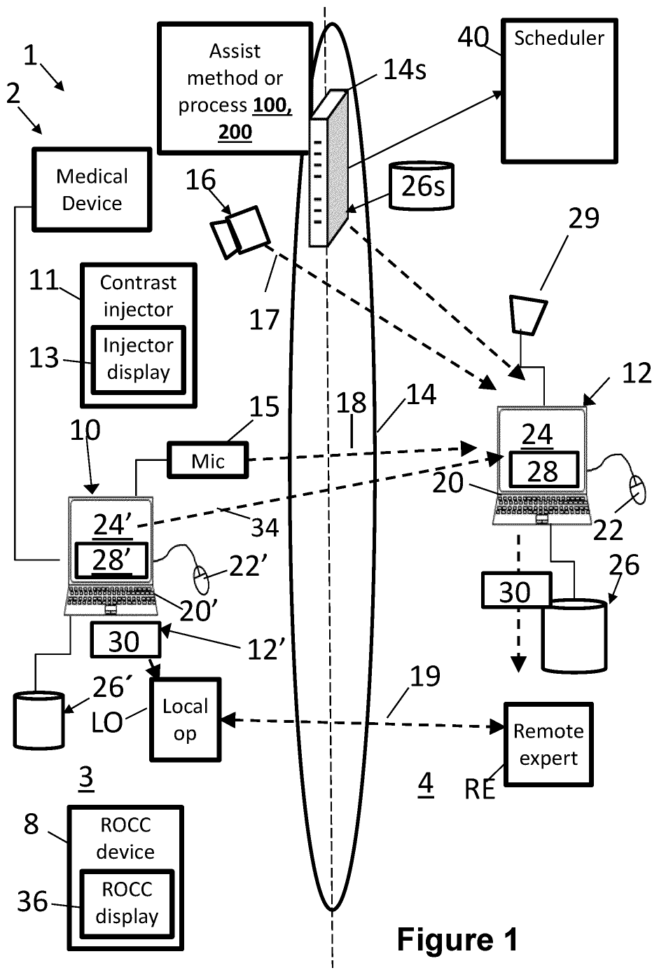

With reference to FIG. 1, an apparatus 1 for providing assistance from a remote medical imaging expert RE (or supertech) to a local technologist operator LO is shown. As shown in FIG. 1, the local operator LO, who operates a medical imaging device (also referred to as an image acquisition device, imaging device, and so forth) 2, is located in a medical imaging device bay 3, and the remote expert RE is disposed in a remote service location or center 4. It should be noted that the “remote expert” RE may not necessarily directly operate the medical imaging device 2, but rather provides assistance to the local operator LO in the form of advice, guidance, instructions, or the like. The remote location 4 can be a remote service center, a radiologist's office, a radiology department, and so forth. The remote location 4 may be in the same building as the medical imaging device bay 3 (this may, for example, in the case of a “remote operator or expert” RE who is a radiologist tasked with peri-examination image review), but more typically the remote service center 4 and the medical imaging device bay 3 are in different buildings, and indeed may be located in different cities, different countries, and/or different continents. In general, the remote location 4 is remote from the imaging device bay 3 in the sense that the remote expert RE cannot directly visually observe the imaging device 2 in the imaging device bay 3 (hence optionally providing a video feed as described further herein).

The image acquisition device 2 can be a Magnetic Resonance (MR) image acquisition device, a Computed Tomography (CT) image acquisition device; a positron emission tomography (PET) image acquisition device; a single photon emission computed tomography (SPECT) image acquisition device; an X-ray image acquisition device; an ultrasound (US) image acquisition device; or a medical imaging device of another modality. The imaging device 2 may also be a hybrid imaging device such as a PET/CT or SPECT/CT imaging system. While a single image acquisition device 2 is shown by way of illustration in FIG. 1, more typically a medical imaging laboratory will have multiple image acquisition devices, which may be of the same and/or different imaging modalities. For example, if a hospital performs many CT imaging examinations and relatively fewer MRI examinations and still fewer PET examinations, then the hospital's imaging laboratory (sometimes called the “radiology lab” or some other similar nomenclature) may have three CT scanners, two MRI scanners, and only a single PET scanner. This is merely an example. Moreover, the remote service center 4 may provide service to multiple hospitals. The local operator controls the medical imaging device 2 via an imaging device controller 10. The remote operator is stationed at a remote electronic processing device 12 (for example, a remote workstation 12).

To provide for contrast-enhanced imaging, a contrast injector 11 is configured to inject the patient with a contrast agent. The contrast injector 11 is a configurable automated contrast injector having a display 13. The user (usually the imaging technologist) loads a vial or syringe of contrast agent (or two, or more, vials of different contrast agent components) into the contrast injector 11, and configures the contrast injector 11 by entering contrast injector settings such as flow rates, volumes, time delays, injection time durations, and/or so forth via a user interface (UI) of the contrast injector 11. The UI may be a touch-sensitive overlay of the display 13, and/or physical buttons, keypad, and/or so forth. In a variant embodiment, the contrast injector 11 is integrated with the imaging device controller 10 (e.g., via a wired or wireless data connection), and the contrast injector 11 is controlled via the imaging device controller 10, including displaying the contrast injector settings in a (optionally selectable) window on the display of the imaging device controller 10. In such an embodiment, the dedicated physical injector display 13 of the contrast injector may optionally be omitted (or, alternatively, the dedicated physical injector display 13 may be retained and the contrast settings displayed at both the dedicated physical injector display 13 and at the imaging device controller 10). In general, the automated contrast injector 11 can employ any suitable mechanical configuration for delivery of the contrast agent (or agents), such as being a syringe injector, a dual-syringe injector, pump-driven injector, or so forth, and may include hardware for performing advanced functions such as saline dilution of the contrast agent, priming and/or flushing of the contrast injection line with saline, and/or so forth.

As used herein, the term “medical imaging device bay” (and variants thereof) refer to a room containing the medical imaging device 2 and also any adjacent control room containing the medical imaging device controller 10 for controlling the medical imaging device. For example, in reference to an MRI device, the medical imaging device bay 3 can include the radiofrequency (RF) shielded room containing the MRI device 2, as well as an adjacent control room housing the medical imaging device controller 10, as understood in the art of MRI devices and procedures. On the other hand, for other imaging modalities such as CT, the imaging device controller 10 may be located in the same room as the imaging device 2, so that there is no adjacent control room and the medical bay 3 is only the room containing the medical imaging device 2. In addition, while FIG. 1 shows a single medical imaging device bay 3, it will be appreciated that the remote service center 4 (and more particularly the remote electronic processing device 12) is in communication with multiple medical bays via a communication link 14, which typically comprises the Internet augmented by local area networks at the remote expert RE and local operator LO ends for electronic data communications. In addition, while FIG. 1 shows a single remote service center 4, it will be appreciated that the medical imaging device bays 3 is in communication with multiple medical bays via the communication link 14.

As diagrammatically shown in FIG. 1, in some embodiments, a camera 16 (e.g., a video camera) is arranged to acquire a video stream or feed 17 of a portion of a workspace of the medical imaging device bay 3 that includes at least the area of the imaging device 2 where the local operator LO interacts with the patient. In other embodiments, a microphone 15 is arranged to acquire an audio stream or feed 18 of the workspace that includes audio noises occurring within the medical imaging device bay 3 (e.g., verbal instructions by the local operator LO, questions from the patient, and so forth). The video stream 17 and/or the audio stream 18 is sent to the remote electronic processing device 12 via the communication link 14, e.g. as a streaming video feed received via a secure Internet link.

The communication link 14 also provides a natural language communication pathway 19 for verbal and/or textual communication between the local operator and the remote operator. For example, the natural language communication link 19 may be a Voice-Over-Internet-Protocol (VOIP) telephonic connection, an online video chat link, a computerized instant messaging service, or so forth. Alternatively, the natural language communication pathway 19 may be provided by a dedicated communication link that is separate from the communication link 14 providing the data communications 17, 18, e.g. the natural language communication pathway 19 may be provided via a landline telephone. In some embodiments, the natural language communication link 19 allows a local operator LO to call a selected remote expert RE. The call, as used herein, can refer to an audio call (e.g., a telephone call), a video call (e.g., a Skype or Facetime or other screen-sharing program), or an audio-video call. In another example, the natural language communication pathway 19 may be provided via an ROCC device 8, such as a mobile device (e.g., a tablet computer or a smartphone), or can be a wearable device worn by the local operator LO, such as an augmented reality (AR) display device (e.g., AR goggles), a projector device, a heads-up display (HUD) device, etc., each of which having a display device 36. For example, an “app” can run on the ROCC device 8 (operable by the local operator LO) and the remote electronic processing device 12 (operable by the remote expert RE) to allow communication (e.g., audio chats, video chats, and so forth) between the local operator and the remote expert.

FIG. 1 also shows, in the remote service center 4 including the remote electronic processing device 12, such as a workstation, a workstation computer, or more generally a computer, which is operatively connected to receive and present the video feed 17 of the medical imaging device bay 3 from the camera 16 and/or to the audio feed 18. Additionally or alternatively, the remote electronic processing device 12 can be embodied as a server computer or a plurality of server computers, e.g. interconnected to form a server cluster, cloud computing resource, or so forth. The electronic processing device 12 includes typical components, such as an electronic processor 20 (e.g., a microprocessor), at least one user input device (e.g., a mouse, a keyboard, a trackball, and/or the like) 22, and at least one display device 24 (e.g. an LCD display, plasma display, cathode ray tube display, and/or so forth). In some embodiments, the display device 24 can be a separate component from the electronic processing device 12. The display device 24 may also comprise two or more display devices. The electronic processor 20 is operatively connected with a one or more non-transitory storage media 26. The non-transitory storage media 26 may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the electronic processing device 12, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a graphical user interface (GUI) 28 for display on the remote operator display device 24. The video feed 17 from the camera 16 can also be displayed on the display device 24, and the audio feed 18 can be output on the remote electronic processing device 12 via a loudspeaker 29. In some examples, the audio feed 18 can be an audio component of an audio/video feed (such as, for example, recording as a video cassette recorder (VCR) device would operate).

FIG. 1 shows an illustrative local operator LO, and an illustrative remote expert RE (e.g., supertech). However, in a Radiology Operations Command Center (ROCC) as contemplated herein, the ROCC provides a staff of supertechs who are available to assist local operators LO at different hospitals, radiology labs, or the like. Each remote expert RE can operate a corresponding remote electronic processing device 12. The ROCC may be housed in a single physical location, or may be geographically distributed. For example, in one contemplated implementation, the remote expert RE are recruited from across the United States and/or internationally in order to provide a staff of supertechs with a wide range of expertise in various imaging modalities and in various imaging procedures targeting various imaged anatomies. A server computer 14s can be in communication with the medical imaging bay 3 and the remote service center 4 with one or more non-transitory storage media 26s. The non-transitory storage media 26s may, by way of non-limiting illustrative example, include one or more of a magnetic disk, RAID, or other magnetic storage medium; a solid state drive, flash drive, electronically erasable read-only memory (EEROM) or other electronic memory; an optical disk or other optical storage; various combinations thereof; or so forth; and may be for example a network storage, an internal hard drive of the server computer 14s, various combinations thereof, or so forth. It is to be understood that any reference to a non-transitory medium or media 26s herein is to be broadly construed as encompassing a single medium or multiple media of the same or different types. Likewise, the server computer 14s may be embodied as a single electronic processor or as two or more electronic processors. The non-transitory storage media 26s stores instructions executable by the server computer 14s.

The medical imaging device controller 10 in the medical imaging device bay 3 also includes similar components as the remote electronic processing device 12 disposed in the remote service center 4. Except as otherwise indicated herein, features of the medical imaging device controller 10, which includes a local electronic processing device 12′, disposed in the medical imaging device bay 3 similar to those of the remote electronic processing device 12 disposed in the remote service center 4 have a common reference number followed by a “prime” symbol, and the description of the components of the medical imaging device controller 10 will not be repeated. In particular, the medical imaging device controller 10 is configured to display a GUI 28′ on a display device or controller display 24′ that presents information pertaining to the control of the medical imaging device 2, such as configuration displays for adjusting configuration settings an alert 30 perceptible at the remote location when the status information on the medical imaging examination satisfies an alert criterion of the imaging device 2, imaging acquisition monitoring information, presentation of acquired medical images, and so forth.

The remote electronic processing device 12 receives various data streams from the imaging bay 3 that enable the remote expert RE to monitor the imaging examination being performed by the local operator LO using the medical imaging device 2. These data streams may include the video stream 17 and/or the audio stream 18 as already discussed. The data streams typically also include a screen mirroring data stream 34 that carries the content presented on the display device 24′ of the medical imaging device controller 10 and presents it on the display 24 of the remote electronic processing device 12, thus providing a live video feed of the display content of the display device 24′ of the medical imaging device controller 10. The screen mirroring data stream 34 may be generated in various ways. In one approach, a video cable splitter such as a DVI splitter, HDMI splitter, or so forth is connected to both send the video signal forming the content of the controller display to the local display 24′ and to provide the screen mirroring data stream 34. In another approach, the screen mirroring data stream 34 may be provided via an auxiliary video output (e.g. aux vid out) port of the imaging device controller 10. In yet another approach, the screen mirroring data stream 34 is generated by screen sharing software 13 running on the imaging device controller 10 which captures a real-time copy of the display 24′ of the imaging device controller 10, and this copy is sent from the imaging device controller 10 to the remote workstation 12. Other approaches are contemplated for capturing a real-time copy of the display 24′ of the imaging device controller 10 which is then sent to the workstation 12 of the remote expert RE as the screen mirroring data stream 34. In a typical workflow for assisting the local operator LO, the remote expert RE will bring up the screen mirroring data stream 34 in a window shown on the display device 24 of the electronic processing device 12, and optionally other data streams from the imaging bay 3 such as the camera video stream 17 may also be shown in other windows of the display device 24 of the electronic processing device 12, thereby providing the remote expert RE with situational awareness of the state and progress of the medical imaging examination being performed by the local operator LO using the medical imaging device 2. The remote expert RE can then assist the local operator LO by providing advice, instructions, or other information or assistance via the natural language communication pathway 19. The communication link 14 allows for transmitting the various data streams 17, 18, 34 from the imaging bay 3 to the remote service center 4. By way of the screen mirroring data stream 34, the remote expert RE can monitor the examination/scan selection dialog screen, scan settings dialog screen, acquisition monitoring dialog screen, among others. The GUI 28′ is included in the screen mirroring data stream 34 and displayed on the remote electronic processing device display 24 at the remote location 4.

The ROCC apparatus 1 further includes or has access to a scheduler 40 (implemented in the server computer 14s) that maintains a schedule of upcoming medical imaging examinations which are to be monitored by the remote expert RE using the remote electronic processing device 12. The schedule maintained by the scheduler 40 is accessible by the remote expert RE via the remote electronic processing device 12 or another link to enable the remote expert RE to have advance knowledge of upcoming imaging examinations he or she will be responsible for assisting. As previously noted, the remote service center 4 may provide service to multiple hospitals, and more particularly the remote expert RE using the remote electronic processing device 12 typically assists multiple medical imaging examinations in the same or (more typically) different imaging bays over the course of a work shift of the remote expert RE. These imaging examinations may overlap in time, thus requiring the remote expert RE to carefully manage his or her time to ensure providing effective assistance to the local operators performing these numerous and possibly concurrent imaging examinations. When handling concurrent imaging examinations, the remote expert RE will need to allocate time efficiently, for example allocating only cursory monitoring time to a “simple” imaging examination for which the local operator is unlikely to need assistance, and more intently monitoring a concurrently performed “complex” imaging examinations that are more likely to run into some difficulty requiring assistance from the remote expert RE.

In similar fashion, the local operator LO may have access to a schedule provided by the scheduler 40 of upcoming imaging examinations to be performed by the local operator LO. As with the remote expert RE, the local operator LO may want to reference this schedule to assess which upcoming examinations are likely to be challenging for the local operator LO. When a complex examination is upcoming, the local operator LO may want to be aware of this so as to take appropriate action ahead of time such as contacting transport personnel to assist with a challenging (e.g. obese or frail) patient, preparing the contrast injector 11 if the complex examination will involve administering contrast agent, or so forth.

However, the schedules provided to the remote expert RE and local operator LO by the scheduler 40 typically provide only high level information about each upcoming imaging examination. For example, the schedule may only identify the imaging modality and examination type. This sparse information makes it difficult or impossible for the remote expert RE and local operator LO to accurately assess the complexity of each upcoming imaging examination for the purpose of efficient preparation and time allocation.

Thus, as disclosed herein, the server 14s performs a method or process 100 for quantifying complexity of an upcoming medical imaging examination to be performed using a medical imaging device 2. The complexities determined for the respective upcoming medical imaging examinations can be variously used, such as to alert the remote expert RE that an upcoming examination has complexities that may require assistance, or to similarly alert the local operator LO that an upcoming examination will present certain challenges. The instructions to perform the method 100 are stored in the non-transitory computer readable medium 26 of the remote electronic processing device 12.

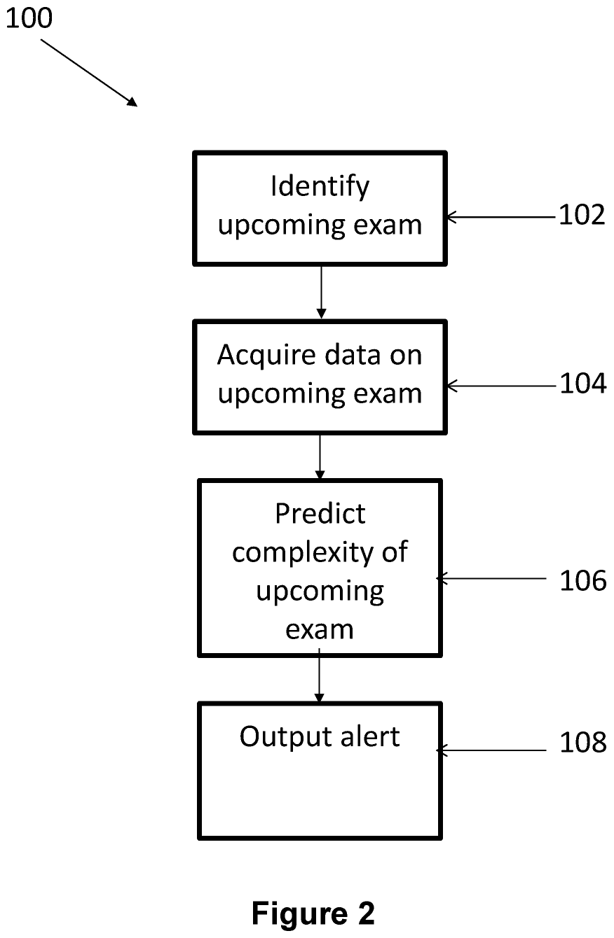

With reference to FIG. 2, and with continuing reference to FIG. 1, an illustrative embodiment of the examination complexity assessment/notification method 100 is diagrammatically shown as a flowchart. To begin the method 100, in an operation 102 the scheduler 40 is accessed in an operation 102 to identify an upcoming medical imaging examination that is to be performed by the local operator LO using the medical imaging device 2.

At an operation 104, data related to the upcoming imaging examination is acquired, for example, from one or more data sources such as HL7 messages, an examination order placed by a referring physician, an examination card, a patient electronic medical record, imaging technician education and experience records, and so forth. The acquired data can include, for example, one or more of a modality of the medical imaging device 2, an imaged anatomy to be imaged in the upcoming imaging examination, specific imaging sequences specified in an imaging examination card for the upcoming imaging examination, and so forth. In another example, the acquired data can include information on historical imaging examinations such as total length, time for each imaging examination phase, contrast agent delivery, patient unloading, idle time, and so forth.

At an operation 106, a complexity of the upcoming imaging examination is determined. To do so, data related to historical imaging examinations is partitioned into one or more classes based on one or more criterion, such as for example, examination type, modality, imaging sequences, patient characteristics, an imaging technologist performing the examination, or a technician's class segmented by experience. Performance statistics for the partitioned historical imaging examinations in each class can be generated. The performance statistics can include, for example, one or more of average total examination time, average time for each phase, average idle time between successive phases, average number of rescans, average number of consultations with a remote expert, average obtained image quality, and so forth. Data related one or more current imaging examinations can be partitioned into one or more classes based on the one or more criterion. The generated performance statistics for the partitioned historical imaging examinations are applied to the one or more current imaging examinations in a common class. The complexity of the upcoming imaging examination is determined based on the applied performance statistics.

In some embodiments, the complexity determination operation 106 can be performed using data related to the patient or the local operator LO. In one example, the complexity of the examination can be determined based on patient characteristics, including but not limited to age, weight, mobility, implants, medical conditions not related to the examination, etc. The complexity determination operation 106 can be adjusted based on these factors.

Similarly, the complexity can be adjusted based on experience of the local operator LO, in general and with regard to the specific examination. Data such as rerun examinations, radiologist feedback, number of similar exams, and training records can be taken into account. The complexity scoring can correspondingly be weighted based on a specific local operator LO. In some embodiments, these two personalized weight factors can be combined, such that a complex patient and a low experience of the local operator LO greatly increases the complexity score of the case.

In another example, the complexity scoring can be used to define a schedule of examinations ahead of time to help optimize the scheduler 40, and then done more real time to account for changes and updates to patient cases, staffing of the local operators LO, staffing of the remote experts RE, and concurrent cases.

In some examples, a prediction of one or more factors contributing to the determined complexity of the imaging examination(s) can be generated. In other examples, the server computer 14s can determine required data to determine the complexity that is missing. For example, an average data value can be assigned for the missing required data, or complexity prediction statistics for coarser examination types that omit that missing information can be generated.

At an operation 108, an alert 30 indicative of the determined complexity of the upcoming imaging examination can be output, for example on the ROCC device 8 for the local operator LO to receive, and/or on the remote electronic processing device 12. for the remote expert RE to receive. Advantageously, the alert 30 is output prior to a beginning of the upcoming imaging examination to forewarn the local operator LO and/or the remote expert RE of an upcoming complex examination. The local operator LO and/or the remote expert RE can provide feedback on the determine complexity at an end of the imaging examination to indicate whether the complexity of the just-completed imaging examination was justified.

While the imaging examination complexity assessment method 100 is described above for a single upcoming medical imaging examination, it will be appreciated that the method 100 can be repeated for each upcoming medical imaging examination at some time ahead of that examination to provide the complexities for some or all upcoming imaging examinations assigned to a given local operator LO or remote expert RE. These results can be variously used. For example, in one approach, any imaging examination whose complexity exceeds a predefined threshold may be flagged as “Complex” in the schedule provided by the scheduler 40. This aspect entails the operation 108 outputting the alert at least to the scheduler 40. In another approach, the method further inspects the schedule to identify any situations in which two (or more) complex examinations (as determined by thresholding of the complexities for example) are assigned at the same time to a single remote expert RE. In such a case, an alert is issued to the remote expert RE of this challenging situation, so that the remote expert can, for example, seek to have one of the complex examinations rescheduled or assigned to another remote expert. In a variant of this embodiment, the detection of concurrent complex examinations assigned to a single remote expert can be sent to the scheduler 40 to perform the rescheduling or remote expert reassignment. In some embodiments, the operation 108 may also entail identifying why a given examination has been determined to be complex.

### EXAMPLE

To perform the examination complexity assessment/notification method 100, the server computer 14s can implement one or more modules stored in the non-transitory storage media 26s. For example, a module for parsing imaging examination information can be provided. The examination information includes patient attributes (e.g., demographics, foreign bodies), technologist attributes (e.g., historical volume, cross modality score, and examination attributes (e.g., body part, contrast)). This module parses all this relevant examination information from different resources including HL7 messages, MR, CT, XR, US modality-based imaging scanners, PACS, RIS, ROCC, and so forth. This module also captures interaction history between local and expert technologists and their associated examination context from the ROCC platform. This module also houses algorithms for data cleaning and data pre-processing.

A module to predict the examination complexity before the examination starts can also be provided. The examination information gets enhanced regularly since the creation of the imaging order. This module updates its prediction on complexity as the new examination information such as examination card, body part becomes available. There is no labelled data for classifying the exams into standard or complex. In this module, the complexity of an examination is measured as a weighted combination of scores obtained from sub-modules. A sub-module can be configured to generate a complexity score using the time taken for activities involved, including a time taken to complete the two activities, preparation and scanning, within an examination are used as the features in this module. Historical data such as contrast, body part, patient demographics etc. are used as features here.

A sub-module can be configured to generate a complexity score based on the idle time between sequences: Idle time between sequences can be extended by lack of patient cooperation, technologist reviewing the images acquired during the current exam, geometry planning etc. The actions in these time periods can be tracked and will be used to classify it into support seeking from expert technologist, patient communication, troubleshooting etc. Analytics on this idle time (number of idle durations in an exam, total idle duration, time spent in reviewing, etc.) will be used as features to classify exams into different levels of complexity.

A sub-module can be configured to generate a complexity score based on the propensity towards repeat/add sequences: Apart from time complexity, the propensity of sequence to be added or repeat sequence can complicate the acquisition process. This repetition or addition of a sequence depends on several factors including patient cooperation, technologist expertise, body part, examination card and their associated sequence names. For a given body part, there are finite set of examination cards to choose from. For each examination card, it is possible to estimate the ratio of repeat/add sequences to the number of total sequences performed. A weighted combination of this ratio is used as the complexity score for a given exam. If the examination is highly standardized such as brain, then most of the sequences are performed right for the first time itself. This in turn, lowers the ratio and hence the weighted combination will also be low.

A sub-module can be configured to generate a complexity score based on the image quality: Motion artifacts such as ghosting reduce the quality of the image obtained. In addition to this, a request for a repeat examination in the corresponding radiology report, previously generated, is an indicator for complex exam. This module uses features like these from historical data to determine the complexity of the exams.

A module to receive feedback from a user can also be provided. A complexity of an examination could be influenced by factors that were not observed from the console screen or camera. To overcome this, survey responses (e.g., How would you rate the examination on a scale of 1-5?) can be gathered from the technologist towards the end of the examination. This response data can be used to improve the predictions. In addition to this, it could also be used to analyze the improvements due to expert user involvement in complex exams.

A module to detect the top contributing factors for examination complexity can also be provided. Like any other data-driven solution, the method 100 needs to earn the trust from local and expert technologists in its recommendations. To address this, a list of top contributing factors to the complexity involved with the image acquisition for a given examination is also provided. This additional information helps in interpreting the nature of complexity involved and thereby help local/expert technologists to appropriately prepare or act for their examination.

A module to recommend local technologist for expert user support can be provided. The increase in complexity often creates delay, which in turn, causes operational disruptions. Recurring operational disruptions cause staff burnout, long patient wait times. To avoid this, the local technologist can directly book an appointment on expert technologist's calendar if the complexity of image acquisition is predicted high. This recommendation engine uses a model-free approach such as epsilon greedy q-learning algorithms. It tracks the user actions based on these recommendations and builds a reward function on the outcomes such as examination completed within the allocated slot, quality of the image obtained etc. These rewards are fed back into the engine for future predictions.

A module to alert the expert technologist in real-time after the examination starts can also be provided. Dynamic factors such as inability to find the vein to inject IV or patient unable to follow the instructions from the local technologist or incidental findings during the scanning can complicate the image acquisition process. In such cases, additional staff support, or add/repeat sequences need to be performed to acquire good quality images. The image acquisition complexity determined using this kind of dynamically evolving factors would be used to alert the expert technologist.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A system for optimizing medical imaging examinations, the system comprising:
one or more processors configured to access one or more data streams of an imaging bay including at least (i) a screen mirroring data stream comprising content presented on a controller display and (ii) a communication pathway connecting the imaging bay and at least some of the one or more processors;
a complexity scoring module executed by the one or more processors and configured to assess a complexity of an upcoming medical imaging examination identified by a scheduler by:
acquiring data related to the upcoming medical imaging examination, the data comprising one or more of time taken to complete preparation and scanning activities associated with the upcoming medical imaging examination, whether contrast is used, what type of contrast is used, body part to be imaged, patient demographics, idle time between sequences, likelihood of repeated or additional imaging sequences, or image quality; and
determining the complexity of the upcoming medical imaging examination based on a weighted combination of a plurality of complexity scores respectively derived from the acquired data; and
an alerting module configured to output an alert indicative of the determined complexity of the upcoming medical imaging examination.

2. The system of claim 1, wherein determining the complexity of the upcoming imaging examinations includes:
partitioning data related to historical imaging examinations into one or more classes based on one or more criterion; and
generating performance statistics for the partitioned historical imaging examinations in each class;
partitioning data related one or more current imaging examinations into one or more classes based on the one or more criterion;
applying the generated performance statistics for the partitioned historical imaging examinations to the one or more current imaging examinations in a common class; and
determining the complexity of the one or more current imaging examinations based on the generated performance statistics.

3. The system of claim 1, wherein the complexity scoring module is further configured to:
generate a prediction of one or more factors contributing to the determined complexity of the upcoming imaging examinations.

4. The system of claim 1, wherein outputting an alert indicative of the determined complexity of the upcoming imaging examinations includes:
outputting the alert indicating that an upcoming imaging exaction will be complex on an electronic processing device operable by a local operator performing the upcoming imaging examination.

5. A non-transitory computer readable medium storing instructions that, when executed by one or more processors, cause the one or more processors to:
access one or more data streams of an imaging bay including at least (i) a screen mirroring data stream comprising content presented on a controller display and (ii) a communication pathway connecting the imaging bay and at least some of the one or more processors;
assess a complexity of an upcoming medical imaging examination identified by a scheduler by:
acquiring data related to the upcoming medical examination, the data comprising one or more of time taken to complete preparation and scanning activities associated with the upcoming medical imaging examination, whether contrast is used, what type of contrast is used, body part to be imaged, patient demographics, idle time between sequences, likelihood of repeated or additional imaging sequences, or image quality; and
determining the complexity of the upcoming medical imagine examination based on a weighted combination of a plurality of complexity scores respectively derived from the acquired data; and
output an alert indicative of the determined complexity of the upcoming medical imaging examination.

6. The non-transitory computer readable medium of claim 5, wherein the acquired data includes one or more of a modality of the medical imaging device, an imaged anatomy, and specific imaging sequences specified in an imaging examination card.

7. The non-transitory computer readable medium of claim 6, wherein the acquired data further includes one or more of information on historical imaging examinations such as total length, time for each imaging examination phase, contrast agent delivery, patient unloading, and idle time.

8. The non-transitory computer readable medium of claim 5, wherein the acquired data is acquired from one or more data sources including HL7 messages, an examination order placed by a referring physician, an examination card, a patient electronic medical record, and imaging technician education and experience records.

9. The non-transitory computer readable medium of claim 5, wherein determining the complexity of the upcoming imaging examinations further includes:
partitioning data related to historical imaging examinations into one or more classes based on one or more criterion; and
generating performance statistics for the partitioned historical imaging examinations in each class;
partitioning data related one or more current imaging examinations into one or more classes based on the one or more criterion;
applying the generated performance statistics for the partitioned historical imaging examinations to the one or more current imaging examinations in a common class; and
determining the complexity of the one or more current imaging examinations based on the generated performance statistics.

10. The non-transitory computer readable medium of claim 9, wherein the one or more criterion include examination type, modality, imaging sequences, patient characteristics, an imaging technologist performing the examination, or a technician's class segmented by experience.

11. The non-transitory computer readable medium of claim 9, wherein the performance statistics include one or more of average total examination time, average time for each phase, average idle time between successive phases, average number of rescans, average number of consultations with a remote expert, and average obtained image quality.

12. The non-transitory computer readable medium of claim 11, wherein the method further includes:
generating a prediction of one or more factors contributing to the determined complexity of the upcoming imaging examinations.

13. The non-transitory computer readable medium of claim 12, wherein the method-instructions further cause the one or more processors to:
determine required data to determine the complexity that is missing; and
assign an average data value for the missing required data.

14. The non-transitory computer readable medium of claim 13, wherein the instructions further cause the one or more processors to:
generate complexity prediction statistics for coarser examination types that omit that information.

15. The non-transitory computer readable medium of claim 14, wherein outputting an alert indicative of the determined complexity of the upcoming imaging examinations includes:
outputting the alert indicating that an upcoming imaging exaction will be complex on an electronic processing device operable by a local operator (LO) performing the upcoming imaging examination.

16. The non-transitory computer readable medium of claim 15, wherein the instructions further cause the one or more processors to:
receive feedback from the LO on the determine complexity at an end of the imaging examination.

17. The non-transitory computer readable medium of claim 16, wherein outputting an alert indicative of the determined complexity of the upcoming imaging examinations includes:
outputting the alert indicating that an upcoming imaging exaction will be complex on a remote electronic processing device operable by a remote expert (RE) monitoring the upcoming imaging examination.

18. The non-transitory computer readable medium of claim 17, wherein the instructions further cause the one or more processors to:
receive feedback from the RE on the determine complexity at an end of the imaging examination.

19. The non-transitory computer readable medium of claim 18, wherein the alert is output prior to beginning of the one or more upcoming imaging examinations.

20. The non-transitory computer readable medium of claim 19, wherein determining a complexity of the upcoming imaging examinations includes:
determining the complexity based on one or more patient characteristics.

21. The non-transitory computer readable medium of claim 20, wherein determining the complexity of the one or more upcoming imaging examinations includes:
determining the complexity based on data related to a local operator (LO) performing the medical imaging examination.

22. The non-transitory computer readable medium of claim 21, wherein determining a complexity of the upcoming imaging examinations includes:
updating a scheduler comprising a schedule of medical imaging examinations based on a determined complexity for each imaging examination on the schedule.

23. A computer-implemented method for optimizing medical imaging examinations, the method comprising:
accessing one or more data streams of an imaging bay including at least (i) a screen mirroring data stream comprising content presented on a controller display and (ii) a communication pathway connecting the imaging bay and a remote terminal;
assessing a complexity of an upcoming medical imaging examination identified by a scheduler by:
acquiring data related to the upcoming medical examination, the data comprising one or more of time taken to complete preparation and scanning activities associated with the upcoming medical imaging examination, whether contrast is used, what type of contrast is used, body part to be imaged, patient demographics, idle time between sequences, likelihood of repeated or additional imaging sequences, or image quality; and
determining the complexity of the upcoming medical imagine examination based on a weighted combination of a plurality of complexity scores respectively derived from the acquired data; and
outputting an alert indicative of the determined complexity of the upcoming medical imaging examination.
